(19) United States
(12) Patent Application Publication
(10) Pub. No.: US 2013/0015654 A1
(43) Pub. Date: Jan. 17, 2013

(54) Air-Tight Push-In and Pull-Out Connector System with Positive Latching

(71) Applicants: Jeffrey Jay Gilham, Sammamish, WA (US); William Oren Wekell, Maple Valley, WA (US)

(72) Inventors: Jeffrey Jay Gilham, Sammamish, WA (US); William Oren Wekell, Maple Valley, WA (US)

(21) Appl. No.: 13/179,563

(22) Filed: Jul. 11, 2011

(51) Int. Cl. F16L 39/02; A61B 5/022

(52) U.S. Cl. 285/124.5

(57) ABSTRACT

The present specification discloses an adapter for coupling a blood pressure cuff to a blood pressure manifold. In one embodiment, the adapter includes an elastomeric hose having a first end and a second end and a first lumen and a second lumen. The first and second lumens define a first air pathway and a second air pathway respectively. A first end connector is integrally formed with the first end and the second end connector is integrally formed with the second end. The first end connector includes a first seal and a second seal and the second end connector includes a third seal and a fourth seal. The first end of the first lumen terminates proximate to the first seal, the second end of the first lumen terminates proximate to the third seal, the first end of the second lumen terminates proximate to the second seal, and the second end of the second lumen terminates proximate to the fourth seal. The seals are comprised of O-rings, compressible wipers, or any other equally compressible, sealing material. Optionally, for a neonate adapter, the third and fourth seals at the second end connector are replaced by an integrated single lumen tube with a neonate cuff attached at its end.

### FIELD OF THE INVENTION

The present invention generally relates to the field of fluid hose connectors or couplings, and more specifically to an air-tight push-in and pull-out connector system capable of mating single and multiple lumen hoses comprising at least one positive latch mechanism.

### BACKGROUND OF THE INVENTION

Conventional fluid hose connecters include tube couplings comprising a male body and a female body which provide for a positive locking capability when joining the male and female bodies together. For example, U.S. Pat. No. 5,984,378 to Ostrander et al describes “[a] quick connector releasibly connects first and second conduits fixedly mounted in first and second, interconnectable connector bodies. The first body has an axially extending tubular first end, a frusto-conical portion, a reduced diameter shaft and an enlarged second end. A pair of angularly inwardly extending latch arms project from the second body to releasibly engage a peripheral edge on the frusto-conical portion of the first body.”

European Patent No. EP-B 8400000051 assigned to Tokai Rubber Industries discloses “a hose connecting structure comprising a hose (10) in which at least the central portion (16) comprises a thermal flexibility resin which is molded by a blow molding method; a mating member (28) which is inserted into the edge opening of said hose, or into which the edge portion of said hose is inserted; a ring-shaped sealing material (14); a first engaging portion (22) which is disposed in the axial direction with a predetermined distance to said sealing material at an edge portion of one of said hose and said mating member; and a second engaging portion (36) which is directly or indirectly engaged with said first engaging portion at the other of the edge portion of said hose and said mating member, wherein said engaging portion of said hose is integrally formed with said hose by the blow molding method and located on the same side wall of said one of said hose and said mating member on which side wall said sealing material rests, wherein the portion of the other said hose and said mating member cooperating with said sealing material is free of a specific adaption.”

U.S. Pat. No. 5,033,777 assigned to Colder Products Company describes “[a] male insert member (12) having a part line free seal member (20) of one piece with the male insert member (12) and method for making the male insert (12) is disclosed. The male insert member (12) is configured for insertion into a female receptacle assembly (14) of a coupling assembly (10). The seal member (20) cooperates with an inner surface of a bore (30) to create a fluid tight seal therewith upon insertion into the bore (30).”

European Patent Application No. EP-A 1520000599 assigned to Fisher & Paykel Healthcare Limited discloses “[a] connector (1) for use with a conduit (3) to supply gases to a user (13) is disclosed. The connector (1) comprises a pair of connectors (22,23), adapted to fit together and swivel relative to one another. More particularly, the connector may comprise a female (36) and male (34) connector assemblies, adapted to be push fit together. The female connector (36) assembly preferably has an extended shoulder and a number of triangular guide slots (43,44), the slots being shaped so as to be wider at one end. The male connector assembly preferably comprises a male portion (37) and a sleeve (38) that when joined, cannot be easily separated by axial or torsional tension applied to either, but may rotate freely relative to each other. The sleeve (38) preferably has a number of guide ridges (41,42) running axially along its outside surface that slidably mate with the guide slots (43,44) on the female connector (36).”

U.S. Pat. No. 4,580,816 assigned to E. R. Squibb & Sons, Inc. describes “[a] tube coupling having a male body, a female body for receiving at least a portion of the male body and at least one peripheral member made from a relatively compressible plastic material molded into a groove within either the relatively rigid male or female body. The peripheral member is disposed to become compressed against a surface of the mating body to form a liquid seal. At least two peripheral members disposed in grooves in a tubular shaped end portion of the male body are made of a compressible plastic and serve as liquid sealing and locking means. The female body comprises a receiving end portion with a tapered interior adapted to receive the male tubular end portion. As the tubular end portion is inserted into the receiving end portion, one of the peripheral members engages the tapered interior surface and becomes compressed there against as the tubular end portion is further inserted. As the tubular end portion is fully inserted a second peripheral member seats itself in a groove in the interior surface of the receiving end portion acting to lock the male and female bodies together. The relatively compressible peripheral members are formed in grooves in the relatively rigid male or female body by insert molding or by simultaneous injection molding of two dissimilar plastics.”

U.S. Pat. No. 4,850,622 assigned to Yokohama Aeroquip Corporation discloses “[a] pipe joint for connecting two pipes to each other has a joint member to which one of the pipes is fixed and provided with a bore formed therein, a socket member fixed to the outer peripheral surface of the joint member and provided at its one end with a flange having an opening and also with an annular space defined by the flange, a retainer ring composed of a plurality of segments each having a tapered surface confronting the opening, the segments being arranged in the circumferential direction and held by an elastic band fitting on the outer peripheral surfaces thereof such that the retainer ring composed of the segments can expand and contract radially, and a male member constituted by the other of the pipes and having a bead portion protruding radially outwardly from the outer peripheral surface of the male member at a position near the end of the male member. The retainer ring has an inside diameter smaller than the outside diameter of the bead so that, when the male member is inserted into the bore in the joint member through the opening, the bead portion produces a wedging action on the tapered surfaces of the segments of the retainer ring so as to radially expand the retainer ring overcoming the force of the elastic band thereby allowing the bead portion to clear the retainer ring. After the bead portion has passed the retainer ring, the retainer ring is contracted radially by the force of the elastic band so as to engage with the rear side of the bead portion.”

U.S. Pat. No. 3,603,621 assigned to Frederick L. Parsons discloses “[a] quick locking and releasing coupling for a pair of members to be coupled together including resilient coupling means projecting from each member to be coupled and snap-locking with the other member to be coupled and a raised cam engageable with the resilient coupling means when they are twisted or rotated relatively to each other to spread the resilient coupling means and release the member with which it was locked.”

The aforementioned prior art connector systems are either not ideal for an air-tight coupling in fail-safe medical applications and/or lack the simplicity of a push-in/pull-out type of coupling that is enhanced by a latch mechanism which does not need to be released by pressing or squeezing to allow the connectors to disengage. Accordingly, there is a need for a quick connect push-in/pull-out connector system that provides air-tight fluidic coupling ideal for medical applications such as non-invasive blood pressure measurement systems. There is also need for a coupling that is capable of mating single and multiple lumen hoses quickly.

### SUMMARY OF THE INVENTION

The present invention is an adapter for coupling a blood pressure cuff to a blood pressure manifold, comprising a hose having a first end and a second end, wherein said hose comprises a first lumen and a second lumen wherein said first and second lumens define a first air pathway and a second air pathway respectively; a first end connector integrally formed with said first end wherein said first end connector comprises a first seal and a second seal and wherein each of said first and second seals have different areas; a second end connector integrally formed with said second end wherein said second end connector comprises a third seal and a fourth seal and wherein each of said third and fourth seals have different areas; and wherein a first end of said first lumen terminates proximate to said first seal, a second end of said first lumen terminates proximate to said third seal, a first end of said second lumen terminates proximate to said second seal, and a second end of said second lumen terminates proximate to said fourth seal.

Optionally, the second end connector is configured to couple to a coupler attached to said blood pressure manifold. The second end connector slidably engages said coupler. The first end connector is configured to the blood pressure cuff. The first end connector slidably engages said cuff. The adapter further comprises a plurality of protrusions positioned on an exterior of said hose. The protrusions are positioned between said first end connector and said second end connector. The first air pathway is configured to direct air from said blood pressure manifold to said blood pressure cuff. The second air pathway is configured to direct air pressure changes to pressure sensors in said blood pressure manifold. At least one of said first, second, third, or fourth seals is an O-ring.

In another embodiment, the present invention comprises an adapter, comprising: an elastomeric member having a first end and a second end, wherein said elastomeric member comprises a first lumen and a second lumen wherein said first and second lumens define a first air pathway and a second air pathway respectively; a first end connector attached to said first end wherein said first end connector comprises a first seal of a first diameter and a second seal of a second diameter and wherein each of said first and second diameters are different; a second end connector attached to said second end wherein said second end connector comprises a third seal of a third diameter and a fourth seal of a fourth diameter and wherein each of said third and fourth diameters are different; and wherein a first end of said first lumen terminates proximate to said first seal, a second end of said first lumen terminates proximate to said third seal, a first end of said second lumen terminates proximate to said second seal, and a second end of said second lumen terminates proximate to said fourth seal.

Optionally, the second end connector is configured to slidably engage with a coupler attached to a blood pressure manifold. The first end connector is configured to slidably engage with a blood pressure cuff. The adapter further comprises a plurality of protrusions positioned on an exterior of said elastomeric member. The protrusions are positioned between said first end connector and said second end connector. The first air pathway is configured to direct air from a blood pressure manifold to a blood pressure cuff. The second air pathway is configured to direct air pressure changes to pressure sensors in a blood pressure manifold. The first seal has the same diameter of said third seal. The second seal has the same diameter of the fourth seal. At least one of said first, second, third, or fourth seal comprises an O-ring.

It is an object of the present invention to provide an air-tight push-in/pull-out type of coupling that does not need to be released by pressing or squeezing to allow the connectors to disengage and provide a positive lock with feedback to the user that the connection has been made. Accordingly a non-invasive blood pressure determination system comprises a monitor or hub coupled to a patient cuff using the connector system of the present invention.

In one embodiment of the present invention the connector system comprises an adapter with a dual lumen hose slidingly engageable with a hub-end coupler on one side and a patient cuff-end coupler on the other. The hose can be an adult or a neonatal hose. In one embodiment, the neonate adapter includes an integrated single lumen tube terminating in a neonate cuff on its second end connector in place of the dual seals. The dual lumen hose allows for one airway to be used to inflate the cuff and the other airway to be used as a sensor air path to enable more accurate measurement of pressure in the cuff.

According to an aspect of the present invention a plurality of elastomeric compression members at ends of a male adapter/connector allow for air-tight engagement when the respective ends are mated with the female cuff and/or hub-end coupler. In one embodiment the compression members comprise O-rings. In another embodiment the compression members comprise wipers.

It is another object of the present invention to provide a positive latching mechanism in addition to the push-in/pull-out coupling. Accordingly, an annular ridge on the male adapter/connector comprises sloping leading and trailing edges. The female cuff-end coupler has an entrance comprising a sloping internal wall or edge. As the male adapter is inserted into the female cuff-end, the leading edge of ridge is pushed over the sloped wall at the female cuff-end entrance. Subsequently, the ridge enters a groove at the female cuff-end such that the groove shoulder prevents an accidental disengagement of the coupling.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 is a block diagram of a non-invasive blood pressure determination system, in which the connector system of the present invention may be employed;

FIG. 2A is an illustration of an expanded view of the hub to cuff connector system in accordance with an embodiment of the present invention;

FIG. 2B is an illustration of a sectioned view of the assembled hub to cuff connector system of FIG. 1 a in accordance with an embodiment of the present invention;

FIG. 3 is an illustration of an embodiment of a hub-end coupler comprising a gage pressure vent tube;

FIG. 4 is an illustration of one connector end of the adapter and the cuff-end coupler in the disengaged position in accordance with an embodiment of the present invention;

FIG. 5 is an illustration of a hose and hub-end connector of a neonate adapter in accordance with an embodiment of the present invention;

FIG. 6A is an illustration of a three dimensional view depicting one connector end of the adapter engaged with the cuff-end coupler and sealed via dual O-rings, in accordance with an embodiment of the present invention;

FIG. 6B is an illustration of a cross-sectional view depicting one connector end of the adapter engaged with the cuff-end coupler and sealed via dual O-rings, in accordance with an embodiment of the present invention;

FIG. 6C is an illustration of a cross-sectional view depicting one connector end of the neonate adapter engaged with the cuff-end coupler and sealed via dual O-rings, in accordance with an embodiment of the present invention;

FIG. 7A is an illustration of a three dimensional view depicting one connector end of the adapter engaged with the cuff-end coupler and sealed via dual wipers, in accordance with an embodiment of the present invention;

FIG. 7B is an illustration of a cross-sectional view depicting one connector end of the adapter engaged with the cuff-end coupler and sealed via dual wipers, in accordance with an embodiment of the present invention;

FIG. 7C is an illustration of a cross-sectional view depicting one connector end of the neonate adapter engaged with the cuff-end coupler and sealed via dual wipers, in accordance with an embodiment of the present invention;

FIG. 8A is an illustration of an oblique cross-sectional view of the positive latching mechanism of the connector system in accordance with one embodiment of the present invention; and,

FIG. 8B is an illustration of a top down cross-sectional view of the positive latching mechanism of the connector system in accordance with one embodiment of the present invention.

### DETAILED DESCRIPTION OF THE INVENTION

The present invention is directed toward an air-tight push-in and pull-out connector system capable of mating single and multiple lumen hoses comprising at least one positive latch mechanism.

In one embodiment, the present invention is directed toward an adapter for coupling a blood pressure cuff to a blood pressure manifold, comprising a hose having a first end and a second end, wherein said hose comprises a first lumen and a second lumen wherein said first and second lumens define a first air pathway and a second air pathway respectively; a first end connector integrally formed with said first end wherein said first end connector comprises a first seal and a second seal and wherein each of said first and second seals have different areas; a second end connector integrally formed with said second end wherein said second end connector comprises a third seal and a fourth seal and wherein each of said third and fourth seals have different areas; and wherein a first end of said first lumen terminates proximate to said first seal, a second end of said first lumen terminates proximate to said third seal, a first end of said second lumen terminates proximate to said second seal, and a second end of said second lumen terminates proximate to said fourth seal.

In one embodiment, the seals are comprised of compressible O-rings. In another embodiment, the seals are comprised of compressible wipers.

Optionally, a neonate adapter includes an integrated single lumen tube terminating in a neonate cuff on its second end connector in place of the dual seals.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

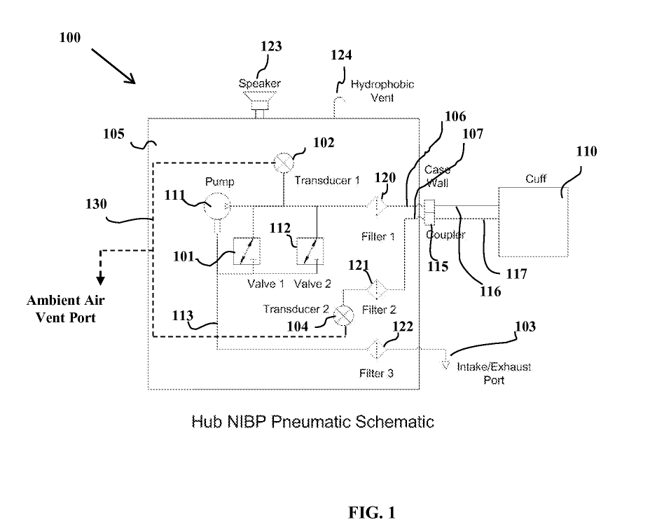

Referring to FIG. 1, a block diagram of a non-invasive blood pressure determination system 100 is shown. The system 100 comprises a monitor or hub 105 coupled to a patient cuff 110 using a connector system/adapter 115 of the present invention. The connector system 115 couples the hub 105 to the cuff 110 using a dual lumen hose, at the cuff end, comprising a first air path 116 and a second air path 117. The air paths 116, 117 respectively correspond to air paths 106, 107 at the hub end. The cuff 110, when attached to the patient's arm, is capable of occluding the brachial artery when fully inflated.

A source of pressure, such as pneumatic pump 111, is used to create air pressure in hose lumen 106 and inflate cuff 110. Redundant bleed valves 101 and 112 are connected to hose lumen 106 leading to cuff 110 at one end and to tube 113 leading to intake/exhaust port 103 at the other. When bleed valves 101 and 112 are activated they are in the ‘closed’ position. When valves 101, 112 are in an ‘open’ position, air flows through them and out of the port 103. When system 100 is pressurized for taking a measurement, the valves 101, 112 are in the ‘closed’ position except when they are briefly opened to lower the pressure in the cuff 110.

When the measurement is finished, valves 101, 112 are opened and the remaining air in the cuff 110 exits the system through port 103. As cuff 110 is deflated using bleed valves 101, 112 via intake/exhaust port 103, the arterial occlusion is gradually relieved. Pressure transducers 102, 104 are coupled by hose lumens 106, 107 respectively to the cuff 110 for sensing the pressure therein. In one embodiment, airway 130 vents a volume of air captured between the manifold and the PCBA (Printed Circuit Board Assembly) that contains transducers 102, 104. This prevents gage pressure vents on the back of transducers 102, 104 from being subjected to pressure variations caused by handling (such as by squeezing) during measurement.

Pressure levels in the hose lumens 106, 107 caused by changes in the pressure of cuff 110 are converted into electrical signals by transducers 102, 104 and communicated to a microprocessor (not shown) for processing. System 100 comprises a microprocessor that is programmed to control the functioning of the system 100 as would be known to persons of ordinary skill in the art. Filters 120, 121 are included in hose lumens 106, 107, respectively, to filter any artifact or noise. In one embodiment, filter 122, included in lumen 113 prevents contaminating elements, such as dust, from entering the system via intake/exhaust port 103 when the pump 111 is actuated. Similarly, filters 120 and 121 prevent contaminants from reaching the transducers 102, 104 and the valves 101, 112. A speaker 123 is optionally included in the hub 105 to provide auditory alarms or blood pressure signals to a caregiver. A hydrophobic vent 124 prevents moisture from affecting the functional elements of the hub 105.

During operation, a pressure signal is created in one lumen, such as lumen 106, greater than the pressure in the other lumen, such as lumen 107, thereby causing at least one of the pressure transducers 102, 104 to measure the change in pressure. This information is then sent to the microprocessor for analysis. The microprocessor uses the results to control (example: regulate, stop, restart, delay) operation of the system 100.

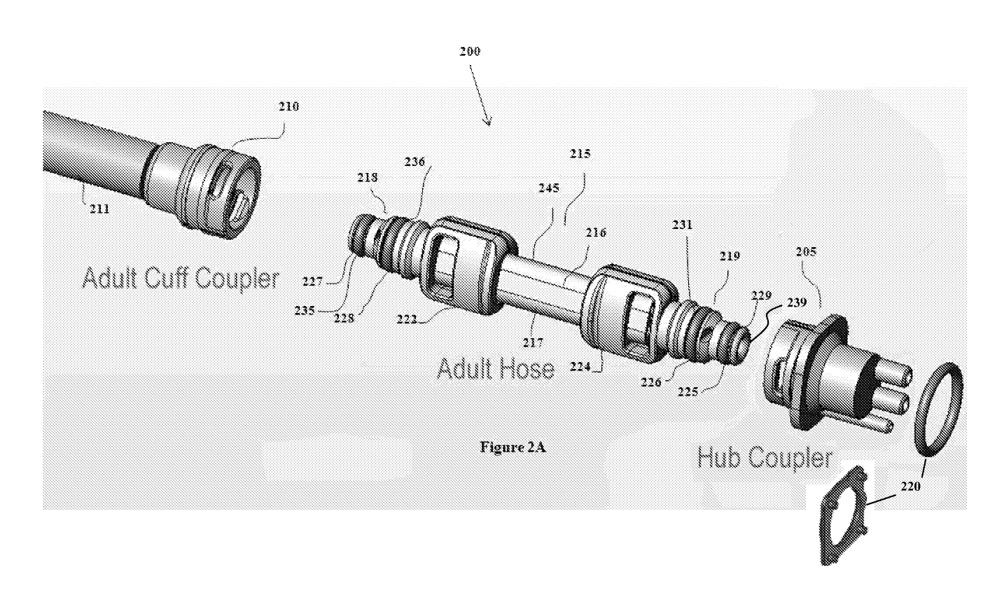

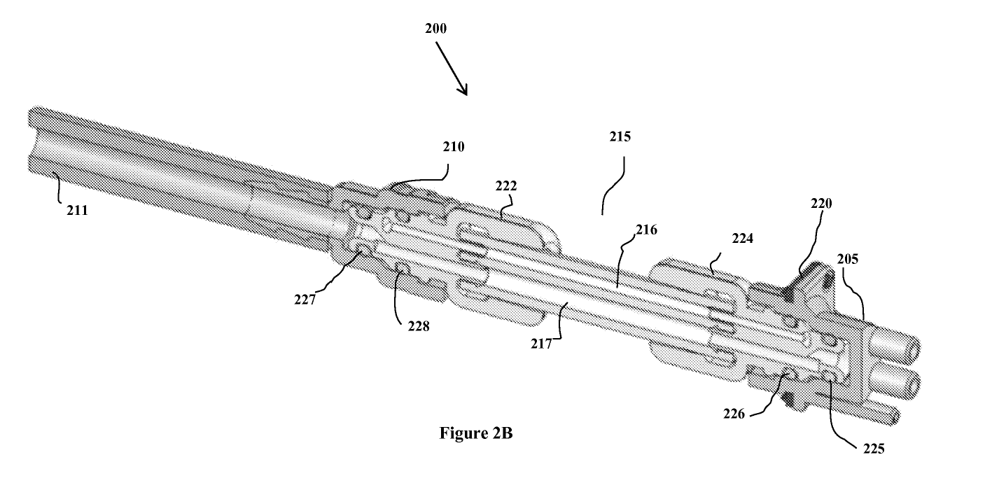

FIGS. 2A and 2B show an expanded and an assembled-sectioned view, respectively, of the hub-to-cuff connector system 200 in accordance with an embodiment of the present invention. The connector system 200 comprises an adapter 215, which in one embodiment forms an adult dual lumen hose, slidingly engageable with a hub-end coupler 205 on one side and a patient cuff-end coupler 210 on the other.

The adapter 215 comprises a first end connector 218, a second end connector 219 and dual lumen hose 245 that laterally defines internal pathway 239. The dual lumen hose 245 in one embodiment is a soft elastomeric extrusion comprising the pathway 239 and extending from the hub-end coupler 205 to the cuff coupler 210. The dual lumen hose 245 is physically divided internally into a first airway 216 used to direct air into and inflate the cuff and a second airway 217 used as a sensor air path. This enables more accurate measurement of pressure in the cuff. Dedicating a single airway for sensor readings enables the system to take measurements during inflation and reduces the number of artifacts introduced into the system.

Handle sections 222, 224 of the first end and second end connectors 218, 219 respectively, are used to handle/manipulate the hose 215 and, in one embodiment, comprise injection molded plastic bodies. In one embodiment, connectors 218, 219 are identical plastic molded parts. The first end connector 218 comprises a plurality of grooves. In one embodiment, the first end connector 218 comprises at least two grooves 235, 236. Similarly, the second end connector 219 comprises a plurality of grooves. In one embodiment, the second end connector 219 comprises at least two grooves 229, 231. In one embodiment, each of the grooves 229, 231, 235, 236 respectively comprises an O-ring 225, 226, 227, 228. Each of these O-rings enables air-tight engagement when the respective first and second end connectors 218, 219 are mated with the cuff coupler 210 and hub-end coupler 205.

In one embodiment, adapter 215 is less than one 1 meter long, while in alternate embodiments it is longer, e.g. less than 2 meters long, less than 3 meters long, less than 4 meters long, between 1 to 2 meters long, between 2 to 3 meters long, between 3 to 4 meters long, or longer than 4 meters. An elastomeric part 220 helps to seal the hub-end coupler 205 to the hub (not shown). In one embodiment, part 220 is rectangular with protrusions to facilitate assembly while in another embodiment part 220 is circular.

In one embodiment, the cuff-end coupler 210 has a piece of tubing 211 with a single air path to allow for simpler construction that is also similar and compatible with cuffs generally available commercially.

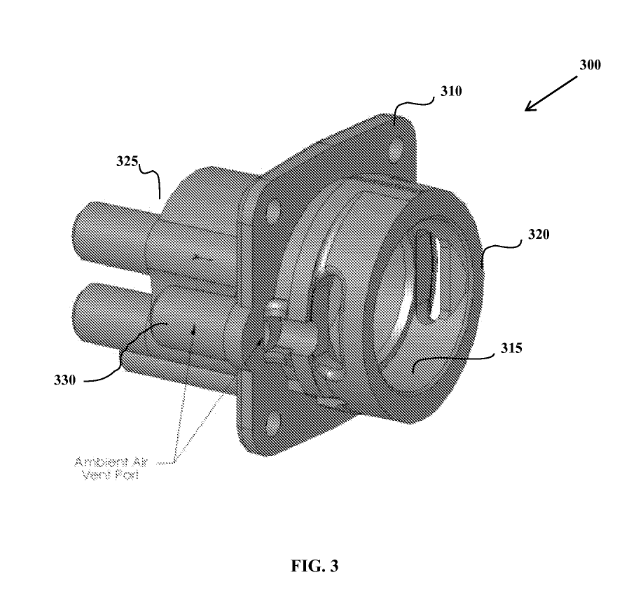

FIG. 3 shows an embodiment of a hub-end coupler 300, similar to the hub-end coupler 205 of FIGS. 2A and 2B. The hub-end coupler 300 comprises a connection plate 310 physically attached and encircling a cylindrical pathway 315 defined by a first port 320 and second port 325. Integrally formed therein is an ambient air vent port 330.

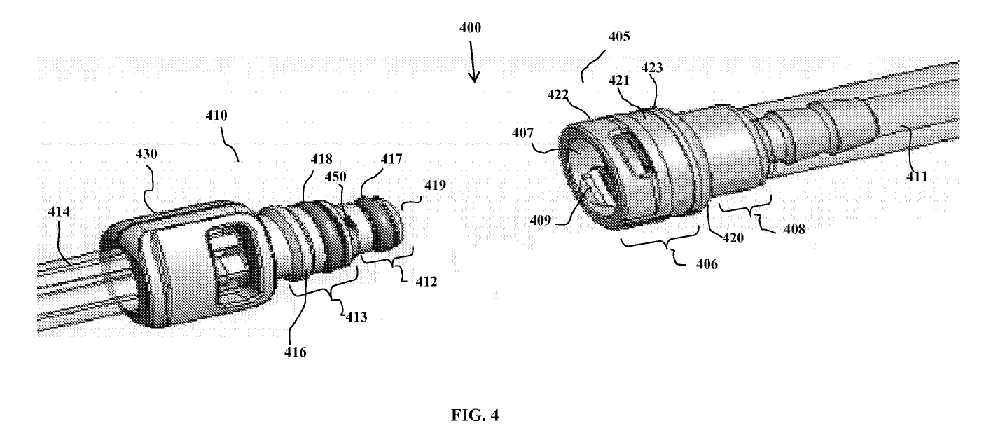

FIG. 4 is an illustration of one connector end of the adapter 410 and the cuff-end coupler 405 in the disengaged position in accordance with an embodiment of the present invention. In one embodiment of the connector system 400, the cuff-end coupler 405 is a female connector and the adapter 410 is a male connector. In one embodiment, the female cuff-end coupler 405 comprises a generally cylindrical, tubular receiving end portion 406 which defines an interior region 407. In one embodiment, the interior of the receiving end portion 406 transitions into another generally cylindrical and tubular portion 408. The diameter of portion 408 is smaller than that of the receiving end portion 406. A distorting collar latch 409, on the interior circumference of the receiving end portion 406, enhances positive engagement when the male adapter 410 is slidingly engaged with the female cuff-end coupler 405. The cuff-end coupler 405 is connected to patient cuff (not shown) by tubing 411.

The outer diameter of the portion 408 is uniform throughout its lateral length. A short sloped step 420 connects the portion 408 to the receiving end portion 406. The outer diameter of receiving end portion 406 is also uniform for most of its lateral length except for a depression or groove 421 that partitions the receiving end portion 406 into forward and backward portions 422, 423 respectively. In one embodiment latch 409 is on the interior circumference of the forward portion 422 of the receiving end portion 406.

The male adapter 410 comprises a hose 414, a handle, 430, and a generally cylindrical, tubular plug end portion 413 that transitions into another generally cylindrical, tubular portion 412. The diameter of the plug end portion 412 is smaller than that of the portion 413. The diameters and lengths of the portions 412 and 413 correspond to the female cuff-end portions 408 and 406 respectively such that the male adapter 410 is adapted to be slidingly inserted into the interior of the female cuff-end coupler 405. In one embodiment, the cylindrical portions 412, 413 include compressible O-rings 417, 418 that act to form an air-tight seal once the two ends of the connector system are engaged. In one embodiment, the O-ring 418 on cylindrical portion 413 is larger in diameter than the O-ring 417 on cylindrical portion 412. In one embodiment, the cylindrical portions 412, 413 include raised shoulder regions on either side of each O-ring 417, 418. These shoulders protect the O-rings from over compression and leak when there is a side pull on hose 414. In one embodiment, the shoulders besides O-rings 417, 418 are about 6 mm and 8 mm in diameter respectively. Strain relief and handle portion 430 has protrusions on to which the hose 414 is forced. In one embodiment, hose 414 comprises two lumens/airways—a first one that breaks the surface 450 of the portion between the O-rings 417, 418 and a second one leading to the very end 419. Persons of ordinary skill in the art should understand that the hose 414 may be single tube/lumen or multiple lumens (such as dual, triple, quadruple and so on) in alternate embodiments.

Also included on the connector end of the adapter 410 is an annular ridge 216 which is generally semi-circular in cross-section and acts as a stop when its leading face, after crossing latch 409, abuts an internal edge (shown in FIG.'s 6A and 6B) of the coupler 405 as the adapter 410 is slidingly engaged within the coupler 405.

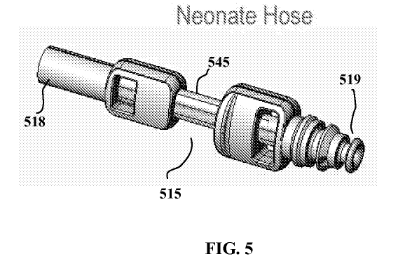

FIG. 5 is an illustration of a hose 545 and hub end connector 519 of a neonate adapter 515 in accordance with an embodiment of the present invention. In one embodiment, the neonate adapter 515 comprises a neonate dual lumen hose 545 that is integral to a cuff (not shown) at end 518 and mates with hub-end coupler 505 at end 519. The adapter 515 comprises substantially all of the aforementioned structural features as adapter 215 of FIG. 2A, except that the first end 518 is integral to the cuff and does not comprise the grooves and O-rings for connection, in accordance with one embodiment. Persons of ordinary skill in the art should note that the neonatal adapters, such as adapter 515 of FIG. 5, are smaller in diameter when compared to adult hoses, such as adapter 215 of FIG. 2A. This is so as the air flow requirement is minimal since the neonatal cuffs are quite small and it is desirable to keep the overall volume of the neonatal system small because the volume of “dead” air in the system attenuates the signal of interest.

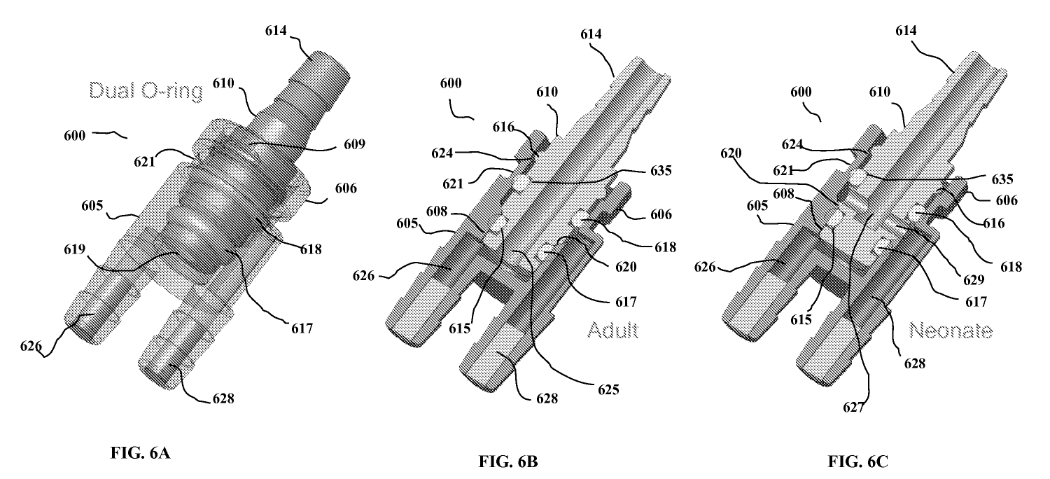

FIGS. 6A and 6B are illustrations of a three dimensional view and a cross sectional view of the connector system 600 depicting one connector end of the adapter 610 engaged with the cuff-end coupler 605 and sealed via dual O-rings 617, 618, while FIG. 6C is an illustration of a cross-sectional view of the connector system 600 depicting one connector end of the neonate adapter 610 engaged with the cuff-end coupler 605 and sealed via dual O-rings 617, 618, in accordance with an embodiment of the present invention. Referring now to FIGS. 6A through 6C, the inner diameter of portion 608 of the cuff-end coupler is uniform throughout its lateral length. A short sloped step 620 connects the portion 608 to the receiving end portion 606. Referring now to FIGS. 4 and 6A-6C simultaneously, the sloped step 420 is formed both on the outer and the inner diameter of the female cuff-end coupler 405 of FIG. 4, while the step 620 is shown only formed on the inner diameter of the female cuff-end coupler 605 of FIGS. 6A through 6C. This difference in depiction of the step illustrates the fact that, in one embodiment, the sloped step design could be only an interior feature of the cuff-end coupler 605, while keeping the outer diameter mostly uniform, or, in another embodiment, could be both an interior as well as an exterior diametric feature of the female cuff-end coupler 405. The outer diameter of receiving end portion 606 is also uniform for most of its lateral length except for a depression or groove 621 that partitions the receiving end portion 606 into forward and backward portions. In one embodiment latch 609 is on the interior circumference of the forward portion of the receiving end portion 606.

Referring now to FIGS. 6B and 6C, persons of ordinary skill in the art should note the structural differences that exist between the neonate male adapter 610 of FIG. 6C and the adult male adapter 610 of FIG. 6B. Thus, while the air path 625 in adult adapter 610 of FIG. 6B is in fluid communication with air path 626 of cuff-end coupler 605, the air path 627 in neonate adapter 610 of FIG. 6C is in fluid communication with air path 628 of cuff-end coupler 605. As shown in FIG. 6C, a short bridge tube 629 forms a communication between the air path 627 and 628. The air path 627 is not formed as a through-bore and therefore does not have fluidic communication with air path 626 of cuff-end coupler 605. This allows a single host connector to service both adult and neonate cuffs through different mutually exclusive paths depending on which hose end adapter is used. Neonate cuffs are much smaller and must be inflated with less flow and pressure to prevent overpressure.

Referring again to FIGS. 6A through 6C simultaneously, for male adapter 610, the outer diameter of the plug end portion contains annular grooves 615, 635 and an annular ridge 216. The annular ridge 616 is generally semi-circular in cross-section and acts as a stop when its leading face, after crossing latch 609, abuts internal edge 624 of depression or groove 621 as the adapter 610 is slidingly engaged within the coupler 605. In one embodiment grooves 615, 635 are generally rectangular in cross-section and occupied by sealing peripheral members 617, 618 formed of compressible elastomeric thermoplastic resin material. In one embodiment the sealing peripheral members 617 and 618 are compressible O-rings with circular cross-sections.

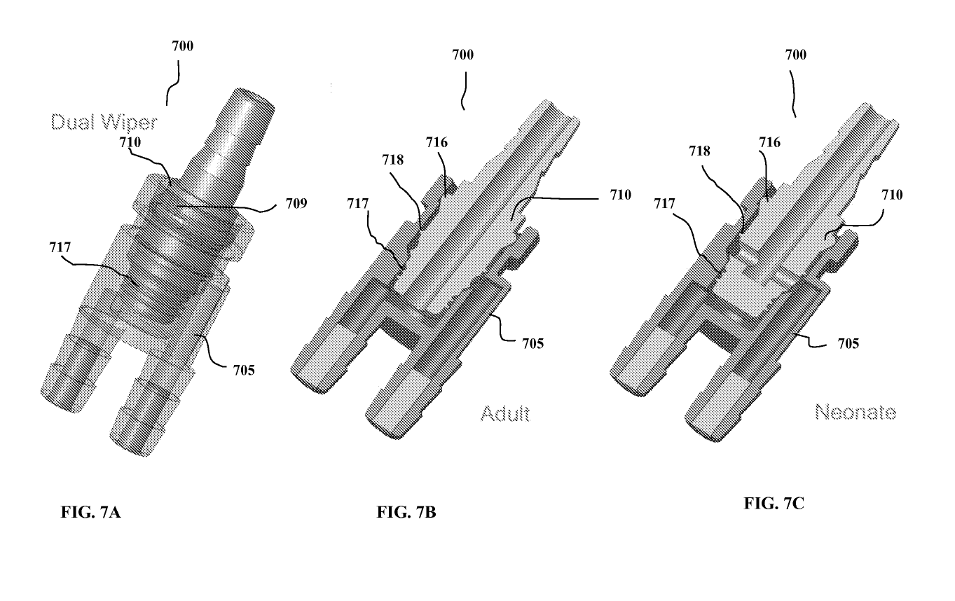

During connecting, as the adapter 610 is inserted into the cuff-end coupler 605, the peripheral member 617 comes in contact with the interior wall of portion 608 while the peripheral member 618 comes in contact with the interior wall of portion 606. The peripheral members 617 and 618 are gradually compressed against the walls as the adapter 610 is fully inserted. Compression related deformation of the peripheral members 617 and 618 against the walls results in an air-tight seal. In one embodiment, the seal is capable of withstanding pressures of up to 6 psi (350 mm Hg) without breaching. The adapter 610 is pushed until the annular ridge 616 has passed the latch 609 and is abutting the internal edge 624. At this point, the sloped step 620 of the cuff-end coupler 605 is also abutting a corresponding parallel sloped edge on the adapter 610. Thus, the annular ridge 616, internal edge 624 and the sloped step 620 act as a stop to further sliding advancement of the adapter 610 into the cuff-end coupler 605. This frees the user from estimating when to stop pushing the adapter 610 into the cuff-end coupler 605. Also, the latch 609 acts as a positive engagement indicator of a completed coupling operation and prevents accidental disengagement of the coupling since the annular ridge 616, that has crossed the latch 609 during pushing, acts as an obstruction against the latch 609 during an outward pull force. As described above, the bodies 605, 610 are quickly and easily connected in one pushing action causing them to be telescopically engaged. To successfully disconnect the two bodies 605, 610 sufficient pulling force must be applied to enable the ridge 616 to again cross over the latch 609—this time in an outward direction. Since the peripheral members 617, 618 (in the form of O-rings) of FIGS. 6A through 6C and the peripheral members 717 and 718 (in the form of wipers, described below) of FIGS. 7A through 7C are compressible, the sufficient pull force causes these peripheral members to give way allowing the bodies to be disengaged. In one embodiment, the system requires 7-12 lb_{F} to disconnect and less than or equal that amount to connect.

Persons of ordinary skill in the art should note that to disengage the bodies 605, 610 only sufficient pulling force is required. No squeezing or pressing action is required on the outer diameter of the portion 606 of the cuff-end coupler 605 or the latch 609.

It should be appreciated that the two O-rings 617, 618 separate the two lumens/airways of the hose 614. In one embodiment, the two O-rings 617, 618 are of different diameters, otherwise the opening in the coupler 605 would chafe and cut the lead O-ring 617 as it was inserted. It should further be appreciated that the present invention covers embodiments in which, at one end, a dual lumen/airway hose of adapter is mated to a single lumen/airway cuff-end coupler and, separately, in which two lumens/airways in the electronics enclosure or hub (such as hub 105 of FIG. 1) mate to two lumens/airways in the hose of the adapter, at the other end, thereby joining patient cuff to the electronics enclosure or hub. It should further be appreciated that the seals disclosed herein can be accomplished using O-rings, elastomeric structures, or any other sealing mechanism.

In another embodiment the sealing peripheral members 617 and 618 of FIGS. 6A through 6C are in the form of wiper-shaped protrusions 717, 718 as shown in FIGS. 7A through 7C. Thus, in the embodiments of the connector system 700 of FIGS. 7A through 7C the sealing function, when male adapter 710 is engaged with female cuff-end coupler 705, is accomplished by the dual wipers 717 and 718. In one embodiment, the wiper 718 is singular while the wiper 717 is a pair. During sliding engagement, annular ridge 716 crosses the latch 709 and acts as an obstruction against the latch 709 when an outward pull force is applied thereby preventing accidental disengagement of the connectors. In one embodiment, wipers 717, 718 are shrunk fit on the male adapter 710 and made of compressible elastomeric thermoplastic resin. Thus, when the male adapter 710 is slid into the female coupler 705, the wipers 717, 718 are compressed by the interior walls of the coupler 705 thereby creating an air-tight sealing effect.

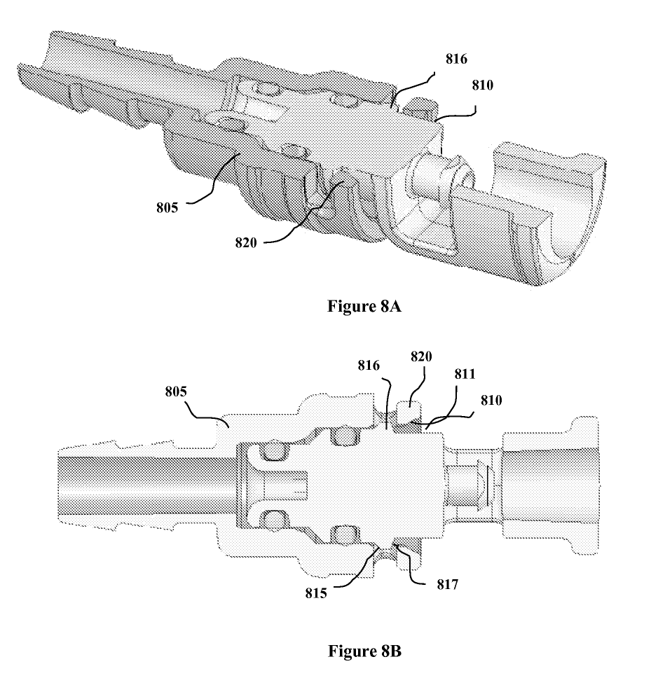

FIGS. 8A and 8B depict positive latching mechanism in accordance with one embodiment of the present invention. Male adapter 810 is inserted into the female cuff-end coupler 805. Annular ridge 816 has a leading edge 815 and a trailing edge 817. The two edges 815, 817 are sloped or have a gradual gradient. The female cuff-end coupler 805 has entry portion 810 also comprising a sloping internal wall or edge 811. As the male adapter 810 is inserted into the female/cuff-end 805, the leading edge 815 of ridge 816 is pushed against the edge 811. Since the leading edge 815 and the edge 811 are appropriately sloped, a sufficient push forces the leading edge 815 to slide over the sloped edge 811. Once the ridge 816 has entered groove 820, the groove shoulder 820 prevents the male hose-end 810 from being inadvertently disengaged. During disengagement, a pull force is applied to the male hose-end 810 sufficient enough to cause the sloped trailing edge 817 of ridge 816 to slide over the groove shoulder 820.

It should be appreciated that the present invention has been described in accordance with multiple different embodiments. Other features, functions, or structures which are equivalent to the ones disclosed herein or obvious alternatives to a person of ordinary skill in the art are intended to be part of, and encompassed by, the present invention.

We claim:

1. A hose and coupler assembly for coupling a blood pressure cuff to a blood pressure manifold, comprising:
a. A hose having a first end and a second end, wherein said hose comprises a first lumen and a second lumen wherein said first and second lumens define a first air pathway and a second air pathway respectively;
b. A first end fitting integrally formed with said first end wherein said first end fitting comprises a first seal and a second seal and wherein each of said first and second seals have different areas;
c. A second end fitting integrally formed with said second end wherein said second end fitting comprises a third seal and a fourth seal and wherein each of said third and fourth seals have different areas; and
d. Wherein a first end of said first lumen terminates proximate to said first seal, a second end of said first lumen terminates proximate to said third seal, a first end of said second lumen terminates proximate to said second seal, and a second end of said second lumen terminates proximate to said fourth seal.

2. The hose and coupler assembly of claim 1 wherein the second end fitting is configured to couple to a coupler attached to said blood pressure manifold.

3. The hose and coupler assembly of claim 2 wherein said second end fitting slidably engages said coupler.

4. The hose and coupler assembly of claim 1 wherein the first end fitting is configured to connect to the blood pressure cuff.

5. The hose and coupler assembly of claim 4 wherein said second end fitting slidably engages said cuff.

6. The hose and coupler assembly of claim 1 further comprising a plurality of protrusions positioned on an exterior of said hose.

7. The hose and coupler assembly of claim 6 wherein said protrusions are positioned between said first end fitting and said second end fitting.

8. The hose and coupler assembly of claim 1 wherein the first air pathway is configured to direct air from said blood pressure manifold to said blood pressure cuff

9. The hose and coupler assembly of claim 8 wherein the second air pathway is configured to direct air pressure changes to pressure sensors in said blood pressure manifold.

10. The hose and coupler assembly of claim 1 wherein at least one of said first, second, third, or fourth seals is an O-ring.

11. The hose and coupler assembly of claim 1 wherein at least one of said first, second, third, or fourth seals is a compressible wiper.

12. A coupler, comprising:
a. An elastomeric member having a first end and a second end, wherein said elastomeric member comprises a first lumen and a second lumen wherein said first and second lumens define a first air pathway and a second air pathway respectively;
b. A first end fitting attached to said first end wherein said first end fitting comprises a first seal of a first diameter and a second seal of a second diameter and wherein each of said first and second diameters are different;
c. A second end fitting attached to said second end wherein said second end fitting comprises a third seal of a third diameter and a fourth seal of a fourth diameter and wherein each of said third and fourth diameters are different; and
d. Wherein a first end of said first lumen terminates proximate to said first seal, a second end of said first lumen terminates proximate to said third seal, a first end of said second lumen terminates proximate to said second seal, and a second end of said second lumen terminates proximate to said fourth seal.

13. The coupler of claim 11 wherein the second end fitting is configured to slidably engage with a coupler attached to a blood pressure manifold.

14. The coupler of claim 11 wherein the first end fitting is configured to slidably engage with a blood pressure cuff.

15. The coupler of claim 11 further comprising a plurality of protrusions positioned on an exterior of said elastomeric member.

16. The coupler of claim 14 wherein said protrusions are positioned between said first end fitting and said second end fitting.

17. The coupler of claim 11 wherein the first air pathway is configured to direct air from a blood pressure manifold to a blood pressure cuff

18. The coupler of claim 16 wherein the second air pathway is configured to direct air pressure changes to pressure sensors in a blood pressure manifold.

19. The coupler of claim 11 wherein said first seal has the same diameter of said third seal.

20. The coupler of claim 18 wherein the second seal has the same diameter of the fourth seal.

21. The coupler of claim 11 wherein at least one of said first, second, third, or fourth seal is an O-ring.

22. The coupler of claim 11 wherein at least one of said first, second, third, or fourth seals is a compressible wiper.

23. An elastomeric coupler integrating a neonate blood pressure cuff for coupling to a blood pressure manifold, comprising:
a. An elastomeric member having a first end and a second end, wherein said elastomeric member comprises a first lumen and a second lumen wherein said first and second lumens define a first air pathway and a second air pathway respectively;
b. A first end fitting integrally formed with said first end wherein said first end fitting comprises a first seal and a second seal and wherein each of said first and second seals have different areas;
c. A second end fitting integrally formed with said second end wherein said second end fitting comprises a single lumen tube ending in said neonate blood pressure cuff; and
d. Wherein a first end of said first lumen terminates proximate to said first seal, a second end of said first lumen terminates proximate to said single lumen tube, a first end of said second lumen terminates proximate to said second seal, and a second end of said second lumen terminates proximate to said single lumen tube.

24. The coupler of claim 23 wherein the first end fitting is configured to slidably engage with a coupler attached to a blood pressure manifold.

25. The coupler of claim 23 further comprising a plurality of protrusions positioned on an exterior of said elastomeric member.

26. The coupler of claim 23 wherein said protrusions are positioned between said first end fitting and said second end fitting.

27. The coupler of claim 23 wherein the first air pathway is configured to direct air from a blood pressure manifold to said neonate blood pressure cuff.

28. The coupler of claim 27 wherein the second air pathway is configured to direct air pressure changes to pressure sensors in a blood pressure manifold.

29. The coupler of claim 23 wherein at least one of said first or second seals is an O-ring.

30. The coupler of claim 23 wherein at least one of said first or second seals is a compressible wiper.
